(19) United States
(12) Patent Application Publication
(10) Pub. No.: US 2026/0294671 A1
(43) Pub. Date: Oct. 1, 2026

(54) MALE CATHETER

(71) Applicant: Kolt Innovations, Inc., Warwick, RI (US)

(72) Inventors: Katelyn Patricia Holt, Warwick, RI (US); Jeanette Numbers, Providence, RI (US); Scott Noh, Cranston, RI (US)

(73) Assignee: Kolt Innovations, Inc., Warwick, RI (US)

(21) Appl. No.: 19/095,430

(22) Filed: Mar. 31, 2025

(51) Int. Cl. A61F 5/453

(52) U.S. Cl. CPC A61F 5/453

(57) ABSTRACT

An external male catheter assembly includes tubular sheath with an outer flexible braided mesh layer and an inner film layer. The tubular sheath has first end to receive a penis and a second end that sealingly connects to a drainage tube. The tubular sheath is expandable along the longitudinal axis and radially outward therefrom so as to grip a penis located therein. The first end of the tubular sheath is rollable upon itself to shorten its length to facilitate receipt of a penis therein. Once located in the rolled up tubular sheath, the first end is unrolled along the penis toward the patient's body to fluidly seal the catheter assembly to the penis in custom fashion. As a result, the tubular sheath stays fixed in place and directs all urine exiting the penis out through the drainage tube for collection.

### BACKGROUND OF THE INVENTION

The present invention relates generally to medical catheters, and more particularly to an external male catheter.

At various times, men can become incontinent or bed-ridden. In such circumstances, it is desirable to collect the urine in a container for subsequent disposal rather than to wear a bulky diaper. Most commonly, a “Texas” condom catheter is used. The Texas catheter includes a condom-like sheath made of silicone rubber or latex. The sheath envelops the penis like a condom but forms a distal opening that can be connected to a hard rubber tube to a collection bag. An adhesive glue is built into the condom to keep the condom in place, but with the slightest movement or weight of the collection bag, the condom may come off.

During urination, the urine passes through the drainage tube or hose for collection in the bag. The bag may be attached to the patient's leg or bed depending upon the circumstances. The sheath may vary in size to improve fit, despite this the sheaths are usually too small or too large. Leakage and skin irritation are common problems. Keeping urine off the skin can prevent skin breakdown and irritation and associated wounds. Additionally, as application of the catheter is commonly performed by healthcare personnel, application can be difficult, cumbersome and awkward resulting in improper placement and function. For ambulatory patients, the leakage problems increase significantly.

Many efforts have been made at addressing these problems but none have proven successful or commercially viable. For example, a common approach has been to utilize a garment with an integral sheath. By elasticizing the garment, improved support and positioning may occur. However, application can become very difficult, particularly with bed-ridden patients, and is susceptible to leaking.

In view of the above, there is a need for a male catheter this external to the body.

There is a need for an external male catheter that is fast and easy to install on the penis of the patient.

There is a further need for an external male catheter that is sanitary by keeping the area clean thereby reducing the chance for infection and other ailments.

Yet a further need is for an external male catheter that is installed in a sealed fashion to the penis and is free of leaks during use.

Another needs is for an external male catheter that is comfortable for the patient to wear.

Still further, there is a need for an external male catheter that is fast and easy to remove from the penis of the patient.

Another need is for an external male catheter that stays on the patient regardless of the position of the patient.

Another need is for an external male catheter that connects well to existing drainage/collection bags.

Another need is for an external male catheter that is dynamically flexible so it moves with the penis regardless of the position of the patient.

Yet another need is for an external male catheter that provides a viable option for urine removal compared to a more invasive and uncomfortable Foley catheter.

Another need is for an external male catheter that can be secured to the thigh or other body part of the patient with a thigh band or similar.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

The present invention provides an external male catheter assembly, configured and arranged for use with a male patient. A tubular sheath includes an outer tubular sheath which is made of an expandable biaxial mesh material. An inner tubular film is secured inside the outer tubular mesh layer to collectively receive a penis therein via an open end. The opposing end tapers to a smaller open end to fluidly communicate with a drainage tube. The outer mesh layer provides the flexibility, stretchability and gripping capability while the inner film layer provides leak proofing and comfort for the patient, among other features discussed herein. The larger open end of the tubular sheath is rollable over itself to adjust its length for installation, sealing and comfort purposes.

To install the catheter of the present invention, the tubular sheath is rolled over itself and the penis is inserted into the open end opposite to the drainage tube connection end. The tubular sheath is unrolled to a location on the shaft of the penis to seal the catheter in place and to optimize the comfort of the patient. Then, the patient uses the catheter and urine is routed through the drainage tube to a collection bag connected thereto. When removal of the catheter is desired, the collection bag is disconnected from the drainage tube and the tubular sheath is rolled up again and then the penis is removed. The catheter can then be cleaned or otherwise disposed of, as desired.

These and other features and advantages will be apparent from a reading of the following detailed description and a review of the associated drawings. It is to be understood that both the foregoing general description and the following detailed description are explanatory only and are not restrictive of aspects as claimed.

In view of the above, an object of the present invention is to provide a male catheter this external to the body.

There is an object of the present invention to provide an external male catheter that is fast and easy to install on the penis of the patient.

Another object of the present invention is to provide an external male catheter that is sanitary by keeping the area clean thereby reducing the chance for infection and other ailments.

Yet another object of the present invention is to provide an external male catheter that is installed in a sealed fashion to the penis and is free of leaks during use.

Another object of the present invention is to provide an external male catheter that is comfortable for the patient to wear.

Still further, another object of the present invention is to provide an external male catheter that is intuitive to use and has a simple removable and is not prone to falling off the penis of the patient.

A further object of the present invention is to provide an external male catheter that stays on the patient regardless of the position of the patient.

Another object of the present invention is to provide an external male catheter that connects well to existing urinary drainage/collection bags.

Another object of the present invention is to provide an external male catheter that is dynamically flexible so it moves with the penis regardless of the position of the patient.

Yet another object of the present invention is to provide an external male catheter that provides a viable option for urine collection compared to a more invasive and uncomfortable Foley catheter, which should not be used for urinary incontinence.

Another object of the present invention is to provide an external male catheter that can be secured to the thigh or other body part of the patient with a thigh band or similar.

### BRIEF DESCRIPTION OF THE DRAWING FIGURES

The novel features that are characteristic of the present invention are set forth in the appended claims. However, the invention's preferred embodiments, together with further objects and attendant advantages, will be best understood by reference to the following detailed description taken in connection with the accompanying Figures in which:

FIG. 1 is a side elevational view of the male catheter of the present invention with tubular sheath sealingly connected to the drainage tube;

FIG. 2 is a cross-sectional view through the line 2-2 of FIG. 1 showing the layers of the device;

FIG. 3 is close-up cross-sectional view of the male catheter of FIG. 2 in the region at the juncture of the drainage tube and the tubular sheath showing the details of the construction of the device;

FIG. 4 is a side elevational view of the male catheter of FIG. 1 with the outer sheath rolled up on itself;

FIG. 5 is a cross-section through the line 5-5 of FIG. 4;

FIGS. 6A-6D show the method for installing the male catheter of the present invention on the penis of a patient;

FIG. 7 shows the male catheter of the present invention installed on a patient with the drainage tube and collection bag secured to the leg of the patient; and

FIG. 8 shows the method for removal of the male catheter of the present invention from the penis of the patient.

### DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

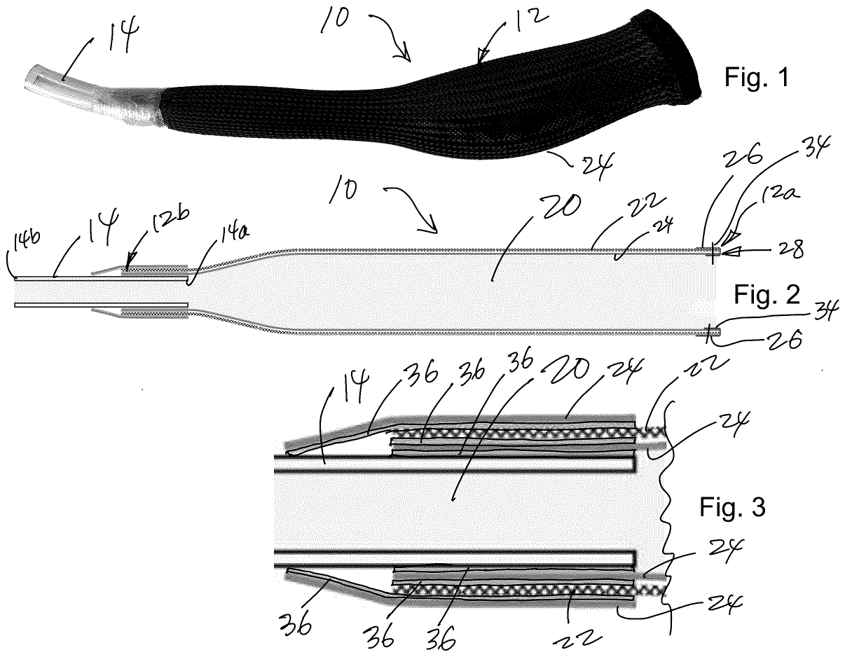

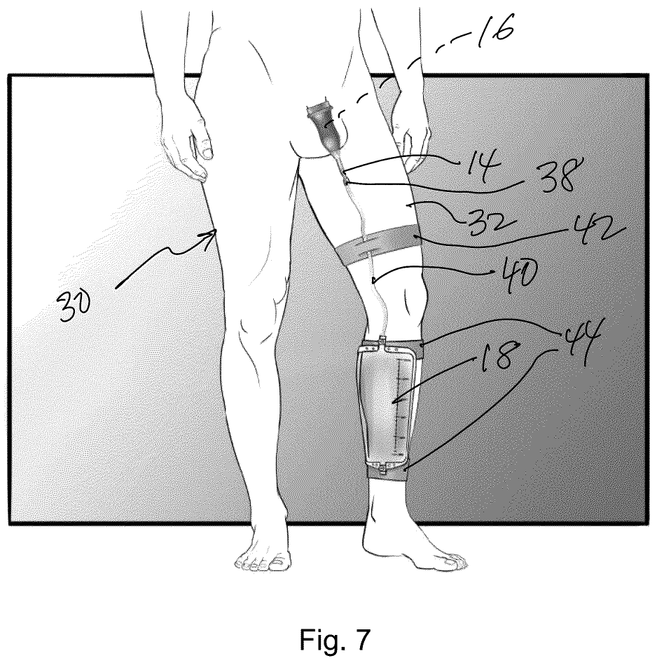

Referring now to the FIG. 1, a side elevational view of the male catheter 10 of the present invention is shown with tubular sheath 12 sealingly connected to a drainage tube 14. The male catheter 10 is fast and easy to securely install to the penis 16 of the user and allows the patient 30, as in FIG. 7, to be ambulatory or, if bed-ridden, urinate without being in a bathroom. The male catheter 10 of the present invention keeps the skin in the area of the penis 16, as in FIG. 7 below, dry and without contact with urine which can cause skin breakdown and infection and associated wounds. As will be described in detail below urine exiting the penis 16 from urination is be collected in a collection bag or drainage bag 18 which may be attached to the leg 32 of the patient 30 or located remote from the patient 30, as in FIG. 7, such as on a wheelchair, table or support surface nearby (not shown). This enables the easy disposal or emptying the collection bag or drainage bag or other container 18. The present construction provides greater patient mobility, which promotes healing and the reduction of provider costs.

Still referring to FIG. 1, the male catheter 10 includes two primary components, namely, a flexible and expandable tubular sheath member 12 and a drainage tube or drainage hose 18. The tubular sheath 12 substantially tubular in configuration with a longitudinal cavity 28 therein and having a first end 12a and a second end 12b. The first end 12a is distal from the drainage tube 18 and is open with a circumferential edge 28. The drainage tube 14 is also tubular with a first open end 14a and a second open end 14b. The opposing second end 12b of the tubular sheath 12 is open but is sealingly secured to first end 14a of the drainage tube 14. Thus, the tubular sheath 12 defines internal cavity 20 that receives a penis 16 on the first end 12a and an opposing second end 12b that terminates in a fluid connection to the drainage tube 14.

FIG. 2 shows the construction and layers of the male catheter 10 of the present invention. The tubular sheath 12 includes a two layer construction that includes an outer sheath 22 that is made of a braided mesh material. For example, a 1.25″ biaxial braided mesh sleeving material is preferably used for the outer sheath 22, such as FLEXO CLEAN CUT but other sizes may be used. This material can expand longitudinally and radially to add comfort while providing gripping for securing the male catheter device of the present invention to a penis. While biaxial braided material is preferred, other sleeving materials can be used and still be within the scope of the present invention.

Still referring to FIG. 2, the second layer of the tubular sheath 12 is an inner tubular film 24. Preferably, the film 24 is a medical grade hydrophilic polyurethane film which is suitable for skin contact, such as MEDIFILM 426 made out of MYLAN. In accordance with the present invention, the inner tubular film 24 is located inside the outer tubular mesh material 22 and is secured at the first end 12a, which is distal to the drainage tube 14. More specifically, as in FIG. 2, a bias binding fabric 26 is used to secure the edges of inner film 24 and the outer mesh material 22 at their first ends at 12a using sewing of thread 34 therethrough. It should be understood that such use of bias binding fabric that is secured by thread is just one way of manufacturing the device and other methods, such as heat sealing, and the like, may be used instead. On the interior side, the bias 26 may overlap 6 mm while the bias 26 overlaps on the outside is 9 mm. The overall diameter of the tubular sheath 12 at rest as in FIG. 2 is 32 mm (about 1.25″) with the overall length of the entire male catheter 10 of the present invention being 260 mm. As a result, the layup of the layers of the outer tubular mesh 22 and the inner tubular film 24 provide the tubular sheath 12 to be installed over a penis 16. The two layers 22, 24 cooperate together to provide a male catheter 10 that provides advantages not found in the prior art. More specifically, the outer tubular mesh 22 is 1.25″ when at rest and is expandable longitudinally and radially when a penis 16 is inserted into the tubular sheath 12 thereby providing an inwardly directed compression and gripping action to the penis 16. This is possible due to the stretchable characteristics of the mesh material 22. As a result, a dynamically flexible sheath 12 that moves with the penis 16 is provided that is far superior to known structures in the industry.

On the other hand, the inner tubular film 24 material provides a comfortable and medically sanitary interface between the tubular sheath 12 and the penis 16. In other words, the inner film 24 provides a medical grade liner to the outer tubular mesh 22. Thus, the double layer structure of the tubular sheath 12 provides stretchability from the mesh 22 and leak proofing, cleanliness, and comfort from the liner 24.

FIG. 3 shows a close-up cross-sectional view of the male catheter 10 of FIG. 2 in the region at the juncture of the drainage tube 14 and the tubular sheath 12 showing the details of the construction of the device at the second end 12b of the tubular sheath 12. More specifically, the second end 12b of dual layer tubular sheath 22 is also open but tapered and secured over a first end 14a of the drainage tube 14. At this second end 12b of the tubular sheath 12, the inner surface of the outer tubular mesh 22 is secured to the outer surface of the inner liner 24 in the region radially surrounding the drainage tube 14 by double-sided tape 36, which is 25 mm. The inner surface of the inner tubular film 24 is, in turn, secured to the outer surface of the first end 14a of the drainage tube 14 also by double-sided tape 36. The drainage tube 14 preferably has an inner diameter of 8 mm and an outer diameter of 11 mm and a length of 60 mm, for example. Finally biocompatible film 24 is also secured about this interconnection location also by double-sided tape 36 to provide an interconnection area of 36 mm As a result, the tubular member 12, with its two layers 22, 24, has one open end 12a to receive a penis 16 while the opposing second end 12b is sealed to the drainage tube 14. It should be noted that the use of double-sided tape 36 is a preferred method for sealingly securing the drainage tube 14 to the tubular sheath 12 but other methods and structure for making this sealed interconnection are also envisioned and are within the scope of the present invention. It should be noted that the dimensions provided herein are just by way of example.

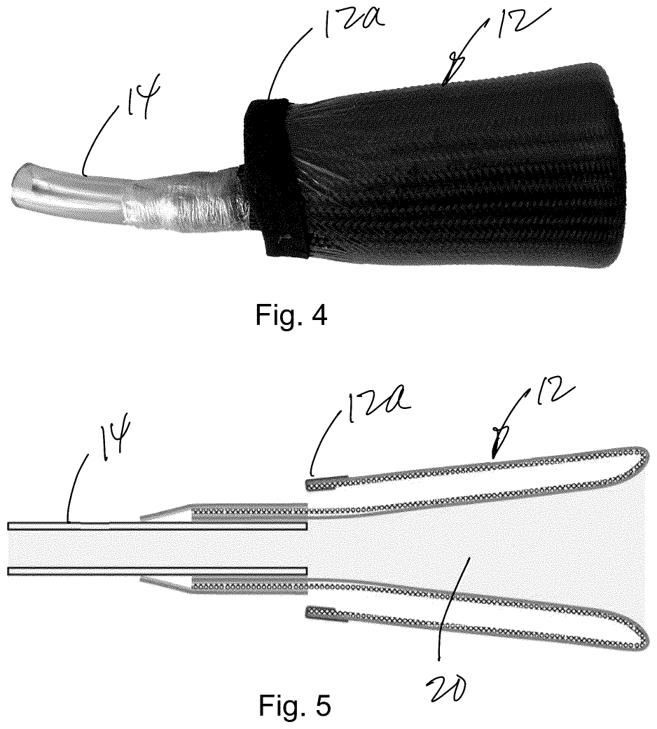

The assembled male catheter 10 of FIGS. 1-3 is flexible and is foldable/rollable upon itself, as seen in FIG. 4, which is a side elevational view of the male catheter 10 of FIG. 1. Such flexibility and rollability is used in connection with the method of installing to and removing the male catheter 10 from the penis 16 of the patient 30. The first end 12a of the tubular sheath 12 is routed over itself so the first end 12a travels toward the drainage tube 14 of the catheter device 10. FIG. 5 shows a cross-section through the line 5 -5 of FIG. 4 to further illustrate the rolling of the tubular sheath 12 over itself. As result, the tubular sheath 12 in its rolled up condition for FIGS. 4 and 5 is ready for receipt of a penis 16 therein. The inner opening is 28 mm while the outer diameter of the rolled up tubular sheath 12 is 43 mm whereby the length of the rolled up tubular sheath 12 is 73 mm thereby shortening the length of the entire catheter device 10 to 135 mm compared to its unrolled condition in FIG. 2 of 260 mm. This results in the catheter device 10 having a diameter of 30 mm at its opening 12 a which is now proximal to the first end 14 a of the drainage tube 14. Due the flexible nature of the tubular sheath 12, it can be rolled up and down easily, which is part of the method of installing and removing the male catheter 10 of the present invention, as described below. It should be noted that the dimensions may be scaled up or down and still be within the scope of the present invention.

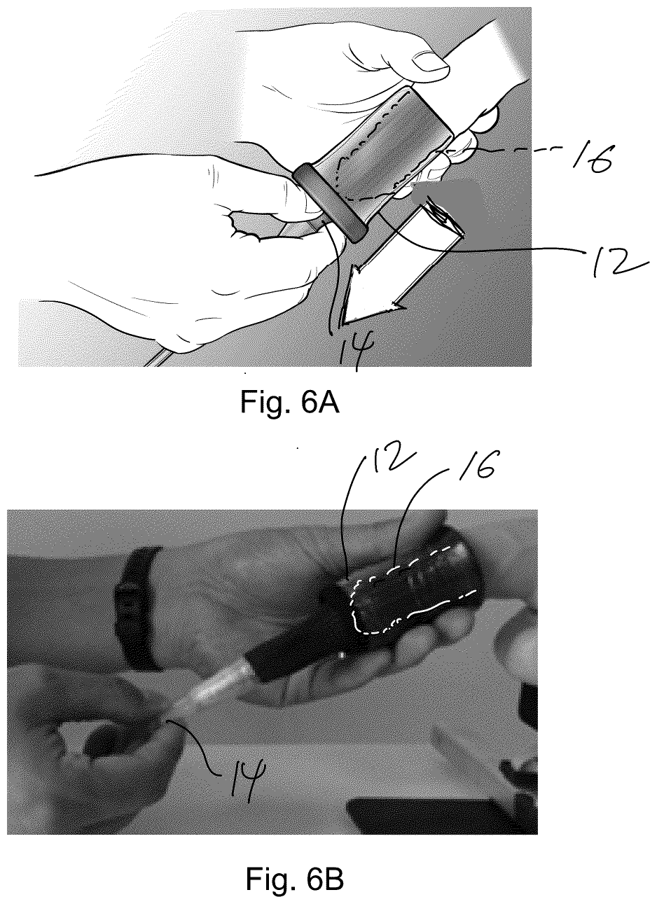

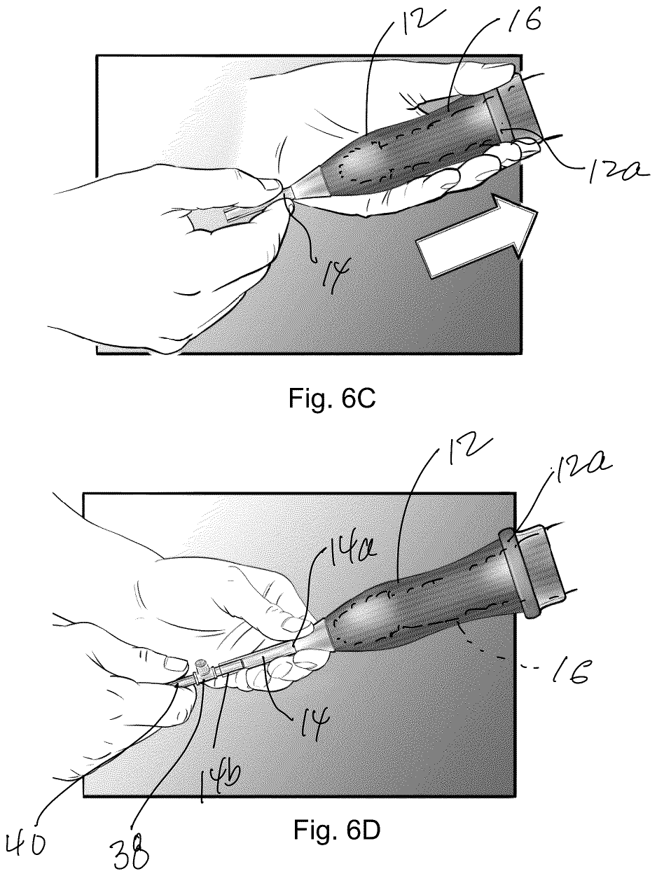

FIGS. 6A-6D show the method for installing the male catheter 10 of the present invention on the penis 16 of a patient 30. Now that the catheter device 10 is rolled up, as in FIGS. 4 and 5, it is ready to be installed on a penis 16. In FIG. 6A, the rolled up catheter 10 is routed over the free end of the penis 16 so the free end is as close as possible to the second end 12b of the tubular sheath 12 at the connection to the drainage tube 14. Then, as seen in FIG. 6B, the first end 12a of the tubular sheath 12 with the fabric secured end 12a/26 is rolled away from the drainage tube 14 toward the patient 30. As this unrolling occurs the tubular sheath 12 flexes outwardly from it natural rest position to thereby gently grip the outer surface of the penis 16. In FIG. 6C, the unrolling of the tubular sheath 12 continues until the rolled portion meets the body of the patient 30 or until a comfortable location is achieved. As in FIG. 6C, the male catheter device 10 of the present invention is now secured in place with the tubular sheath 12 gripping the penis 16 a gentle sealed fashion at a location such as the shaft of the penis 16 without interaction with the head of the penis making it equally effective for circumcised and uncircumcised penises 16. Since no adhesive is used to secure the catheter 10 in place, it is more comfortable and is simpler and easier to install and use.

Turning now to FIG. 6D, the usual interconnections are attached to the second end 14b of the drainage tube in universal fashion to connect the device to an appropriate collection bag or the like. For example, the second end 14b of the drainage tube 14 is fitted with an interconnect 38, such as a tube/hose barb, a Luer lock or other well known tube couplings now known or later developed. As such, a desired tube coupling mechanism 38 may be secured to the second end 14b of the drainage tube 14 for interconnection to the typical collection tube 40 which is routed to a collection bag 18.

FIG. 7 shows installation of the male catheter 10 of the present invention in context to the entire body of the patient 30. The catheter 10 is secured in place to the penis 16 as described above while the collection tube 40 is secured in place using a leg band 40, or the like, which in turn feeds the urine to a leg collection bag 18, which is also strapped to the patient's leg 32 by a bands 44. In such a leg bag application, the collection tube 40 would be of a length appropriate to reach the collection bag 18 and its relatively close location on the leg 32. On the other hand, a longer length collection tube 40 from the drainage tube 14 would be used where the collection bag 18 is located further away, such as being attached to a patient's bed or wheelchair (not shown). Further, the tube 114 may include an occlusion device or clip (not shown) for selectively closing flow there through when changing and emptying the collection bag 18.

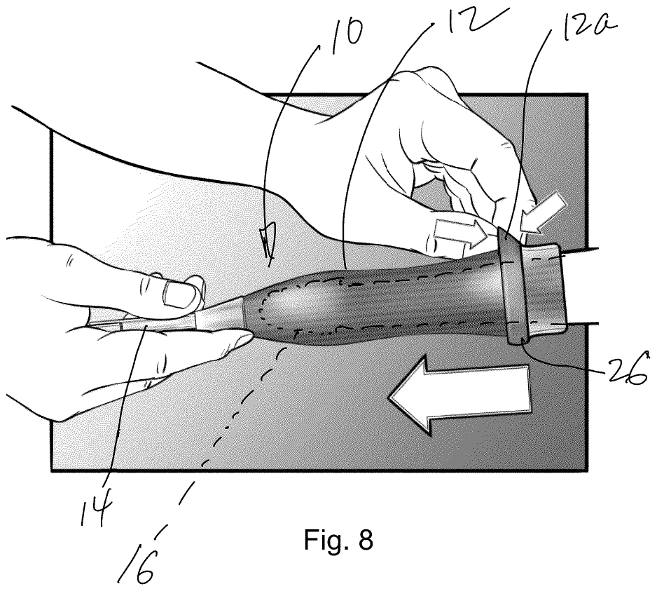

Turning now to FIG. 8, the removal of the catheter 10 of the present invention is shown. When the catheter 10 is no longer needed or needs to be cleaned, it needs to be removed quickly and easily. The second end 12a of the male catheter 10 is held in place, such as with the assistance of holding the drainage tube 14 connected to the tubular sheath 12. The first end 12a of the tubular sheath 12 is gripped at the fabric biasing 26 and is pulled toward the drainage tube 14 and away from the patient 30. Such action rolls back up the tubular sheath 12 while still on the penis 16 back to a rolled up condition similar to what is seen in FIGS. 4 and 5. Once the rolling up of the tubular sheath 12 reaches the condition of FIGS. 4 and 5, there will be less overlap of the tubular sheath 12 over the patient's penis 16 to permit easy removal of the catheter 10 from the penis 16. The catheter 10 of the present invention can be cleaned or discarded, depending on if it is a daily use external catheter or a durable external catheter. When ready for use again, the catheter 10 is rolled back up to the condition in FIGS. 4 and 5 to start the installation process again.

It should also be noted that the male catheter 10 of the present invention could also monitor strict ‘I&Os’ on male patients 30. Such monitoring is important for seeing how much a patient 30 urinates, for example, if they were given a diuretic for congestive heart failure. It is also important for a male patient 30 that may be incontinent to have accurate output measurements. It can be used during surgery in a sterile environment because the male catheter 10 can be offered sterilized and in sterile packaging, but the male catheter 10 does not need to be sterile for the medical units because the male catheter 10 is an external catheter and will not be introducing bacteria into the body like other internally invasive catheters, such as a Foley catheters. Accordingly, the male catheter 10 decreases Catheter-Associated Urinary Tract Infections (CAUTIs) in patients 30 and offers a safe effective alternative to an internal catheter. CAUTIs cause urosepsis, the loss of patient lives and also cost the healthcare millions of dollars.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. An external male catheter assembly, configured and arranged for use with a male patient, comprising:
an outer tubular sheath having a first open end and a second open end with a longitudinal axis and an inner diameter; the outer tubular sheath being expandable along the longitudinal axis and radially outward therefrom; the inner diameter of the outer tubular member being smaller when at rest compared to when the outer tubular member is radially expanded outwardly;
an inner tubular film having a first open and a second open end; the first open end of the inner tubular film being secured to the first open end of the outer tubular sheath and defining an entry port for receipt of a penis therethrough; the outer tubular sheath and inner tubular film defining a tubular body portion having a length, a first end and a second end configured for receiving a penis therein;
a drainage tube having a first end and a second end with an outer surface;
the second open end of the outer tubular sheath being secured to the second open end of the inner tubular film; both the second open end of the outer tubular sheath and the second open end of the inner tubular film being both collectively sealingly secured to the outer surface of the drainage tube proximal to the first end of the drainage tube thereby sealing liquid flow through the tubular body potion and out through the drainage tube;
the tubular body portion having a smaller diameter when a rest than a diameter of a penis whereby routing the tubular body portion over a penis provides a radially inward gripping of the penis thereby sealing the penis in the tubular body portion;
wherein the first end of the tubular body portion being rollable over itself to adjust the length of the tubular body portion and to seal the tubular body portion to the penis.

2. The assembly of claim 1, wherein the outer tubular sheath is made of a biaxial braided mesh material.

3. The assembly of claim 1, wherein the drainage tube is flexible vinyl tubing.

4. The assembly of claim 3, further comprising a collection bag fluidly connected to the drainage tube.

5. The assembly of claim 1, further comprising:
a lock attached to drainage tube.

6. The assembly of claim 1, wherein the film is a biocompatible liquid tight film.

7. The assembly of claim 1, wherein the portion of the tubular body member that has been rolled over itself is positioned at a location along a length of the penis.

8. The assembly of claim 1, wherein the first end of the outer tubular sheath and the first end of the inner tubular film are secured to each other by a fabric sewed thereto.

9. The assembly of claim 1, wherein the second end of the outer tubular sheath and the second end of the inner tubular film are secured to the outer surface of the first end of the drainage tube by double-sided adhesive tape.

10. A method of using an external male catheter assembly configured and arranged for use with a male patient, comprising the steps of:
providing a tubular sheath having a first open end and a second end defining a cavity for receipt of a penis; the tubular sheath being flexible along its length and radially flexible;
providing a drainage tube having a first end and a second end; the first end of the drainage tube sealingly connected to and in fluid communication with the second end of the tubular sheath; the second end of the drainage tube providing an fluid exit port;
rolling the first open end of the tubular sheath over itself toward the second end of the tubular sheath thereby providing an entry port for a penis;
inserting a penis into the entry port;
unrolling the first open end away from the second open end of the tubular sheath leaving an overlapping portion of the tubular sheath over itself;
fluidly sealing the penis in the cavity;
wherein urine exiting the penis is directed out through the fluid exit port.

11. The method of claim 10, wherein the outer tubular sheath is expandable along a longitudinal axis and radially outward therefrom.

12. The method of claim 10, wherein the tubular sheath includes an outer tubular flexible braided mesh member and an inner tubular film member laid up to each other.

13. The method of claim 10, wherein the tubular sheath has a smaller diameter when a rest than a diameter of a penis whereby routing the tubular sheath over a penis provides a radially inward gripping of the penis thereby sealing the penis in the tubular body portion; and wherein rolling of a portion of the tubular sheath adjusts the length of the tubular sheath and to further seal the tubular body portion to the penis.

14. The method of claim 10, further comprising the steps of:
providing a collection bag;
routing fluid through the fluid exit port of the drainage tube into the collection bag.
